(19) United States
(12) Patent Application Publication
(10) Pub. No.: US 2005/0015044 A1
(43) Pub. Date: Jan. 20, 2005

(54) Catheter and method for producing the same

(76) Inventors: Herbert Harttig, Altrip (DE); Reinhold Buck, Alleshausen (DE)

Correspondence Address: Roche Diagnostics Corporation, 9115 Hague Road, PO Box 50457, Indianapolis, IN, 46250-0457, US

(21) Appl. No.: 10/488,135

(22) PCT Filed: Aug. 30, 2002
(86) PCT No.: PCT/EP02/09686

(30) Foreign Application Priority Data
Aug. 30, 2001 (SE) 0102879-4

(51) Int. Cl. A61M 1/00

(52) U.S. Cl. 604/29

(57) ABSTRACT

The present invention concerns a catheter with a hollow fiber membrane (10) bent essentially in U-shape or such a hollow fiber membrane (10) comprising two legs (11, 13) and a bent section (15). According to the invention the hollow fiber membrane (10) is heat-treated and/or treated with at least one solvent in its bent section (15). The catheter according to the invention has a small overall diameter, is simple to manufacture and easy to implant and explant. The bent section (15) does not kink so that the lumen remains open. The present invention also concerns a method for producing such a catheter or such a hollow fiber membrane.

The present invention concerns a catheter according to the preamble to claim 1 and a method for producing such a catheter according to the preamble to claim 18.

A generic catheter for microdialysis is known from WO 99/41606. This catheter has one inlet channel and one outlet channel made of a semipermeable membrane for a dialysis fluid which are arranged in a microchip. Both channels are connected together by a hairpin-shaped section which protrudes from the microchip and through which mass exchange takes place between dialysis and body fluid. The outlet channel is integrally connected to an analytical unit that is also embedded in the microchip for monitoring the composition of the dialysis fluid after the mass exchange. This analytical unit is composed of a reservoir containing a reference substance, a reference electrode or ion-sensitive electrode and a sensor which is for example an ion-sensitive field effect transistor (ISFET). A problem with this embodiment which only has a hollow fiber membrane is that it can only be implanted in tissue by complicated surgical procedures and also has to be surgically removed for explantation since if it is simply pulled out there is a risk that at least parts of the membrane may remain in the body or tissue.

Another catheter is described in WO 98/44978. This catheter has an inner tubular part which is surrounded by an outer tubular part in the form of a hollow fiber membrane. The inner tubular part surrounds an outlet channel. The outer wall of the inner tubular part and the inner wall of the outer tubular part border an inlet channel. In addition spacer means are provided which brace the inner wall of the outer tubular part against the outer wall of the inner tubular part. However, the assembly of such a catheter is very complicated because it is much more difficult to insert an inner tubular part with spacers into the outer tubular part than is the case with a smooth tube.

Another catheter for microdialysis is disclosed in DE 33 42 170 C2. This catheter has a pipe-shaped or tubular dialysis membrane or a hollow fiber membrane accommodated in a metal housing. However, the manufacture of such a catheter is complicated because the dimensions needed require that a very fine tube has to be very exactly inserted into a housing. In addition the tube has a tendency to lean against the inner wall of the housing and thus block a part of the membrane resulting in unpredictable exchange performance.

A problem with all known catheters in which concentric tubes are inserted into one another is that the fluid contact and in particular the interspace between the outer and inner tube or the hollow fiber membrane and the inner tube is complicated and requires a relatively large dead volume. Dead volumes result in longer time periods (dead time) between a change in concentration and the availability of a corresponding sample in a measuring device and should thus be minimized. Furthermore, since there are usually fine-pored layers inside the hollow fiber membrane which are essential for the separation, there is always a risk that this separating layer will be damaged when components are inserted into the hollow fiber membrane.

Hence the object of the present invention is to provide a catheter of the above-mentioned type which is uncomplicated to manufacture and can be simply implanted and explanted.

The object is achieved by a catheter having the features of claim 1 and a method having the features of claim 18.

Hence the catheter according to the invention has a hollow fiber membrane bent by about 180°, i.e. approximately U-shaped, where the two legs of the U are as close together as possible. This would usually kink the hollow fiber membrane which would interrupt that passage of liquid through the lumen. It has surprisingly turned out that the structure of the hollow fiber membrane can be changed, at least in this area, by the anisotropic application of heat or solvents in such a manner that the hollow fiber membrane can be bent by about 180° without kinking i.e. without the lumen closing.

Subsequent investigations showed that this is due to the anisotropic effect of heat or solvents. The action of heat or solvents softens the membrane material. The surface or interfacial tension of the membrane material shrinks the pores. This is manifested macroscopically in a change in their dimensions i.e. the membrane becomes shorter, thinner and denser. Since heat or solvents act anisotropically i.e. from one side, the hollow fiber membrane is bent. The recovery forces of the part of the membrane in the bending area that is not affected by heat or solvent are overcome by external mechanical forces to achieve a 180° bend. Although the lumen is narrowed by the 180° bend it nevertheless remains open.

Furthermore it is advantageous that the connections of the catheter according to the invention are in fact two tubes that can be simply connected. The dialysis fluid enters one end of the hollow fiber membrane and emerges again at the other end.

The catheter for microdialysis according to the invention has a very small overall diameter. Hence the catheter according to the invention is not only very simple to implant and explant, but is also much more comfortable to carry and has a considerably reduced dead volume compared to the prior art.

The method according to the invention is characterized in that the hollow fiber membrane, at least in the area to be bent, is heat treated or treated with at least one solvent. The anisotropic application of heat or anisotropic treatment with at least one solvent changes the structure of the hollow fiber membrane in such a manner that it can be bent into an approximately U-shape without kinking or closing the lumen with a minimal overall diameter of the catheter. In this connection it is particularly advantageous that the hollow fiber membrane only has to be treated from outside in order to manufacture a catheter for microdialysis according to the invention. It has also turned out that it is not necessary to support the lumen as in the prior art by for example filling metallic tubes with sand in order to bend them. Furthermore it is no longer necessary to insert a tube or other part inside a hollow fiber membrane or to insert a membrane tube into a housing. There is also no longer a risk of damaging the inner separating layer of the hollow fiber membrane.

Advantageous further developments result from the subclaims. The distance (a) between the two legs of the hollow fiber membrane should preferably be less than 50% of the diameter (d) of the hollow fiber membrane. Alternatively or in addition, the overall diameter of the catheter should advantageously be less than 2.5 times the diameter of the hollow fiber membrane. This results in the smallest possible size of the structure and a particularly small overall diameter of the final catheter. In addition the U-shaped section of the hollow fiber membrane is preferably bent by 180°±5° and can be reinforced to improve its tensile strength. The use of a reinforcement also further simplifies explantation since it further reduces the risk of losing the membrane or parts of the membrane. The reinforcement can for example be a wire, a thread and/or a braid which for example runs parallel to the two legs and is connected, glued or welded to them. The reinforcement can be made of a metal, a metal alloy or a plastic. Wires made of high-grade steel, a noble metal or monofil polymer fibers, threads made of polymer fibers or braids made of several thin metallic filaments are very suitable.

In the area where the dialysis fluid is fed in and/or discharged, the catheter according to the invention can have a zone that is impermeable to liquid which can also be used as a shaft for connecting the ends.

The hollow fiber membrane can also have at least one substance that absorbs radiation such as dyes and/or pigments at least in selective places in order to increase radiation absorption and thus heat uptake when heat is applied by electromagnetic irradiation.

In the manufacture of the catheter according to the invention the solvent effect is preferably achieved by applying a small drop of a suitable solvent or solvent mixture. The size of the drop depends on the diameter and wall thickness of the hollow fiber membrane. The volume should be sufficient to fill up the pore structure of the membrane over a length corresponding to one to one and a half times the diameter and ca. 50% of the membrane circumference. Suitable solvents are those which dissolve or soften the polymer of the membrane and make it shrink e.g. dimethylformamide, dimethylsulfoxide, dimethylacetamide or tetrahydrofuran. Chlorohydrocarbons such as trichloroethane are not preferred for environmental reasons. Solvents or solvent mixtures that can be easily removed again by evaporation such as mixtures of isopropanol and tetrahydrofuran are particularly preferred.

In the production of the catheter according to the invention it has proven to be particularly suitable to apply heat by direct contact with an electrically heated wire. The introduction of heat by electromagnetic radiation is also possible for example by laser, microwave or high-frequency radiation provided that the radiation is absorbed by the hollow fiber membrane at least in the bent section.

Commercial dialysis hollow fibers are suitable as a hollow fiber membrane, for example those made of polyamides, polyamide S, polyarylether sulfones, polymethacrylate, polysulfones or polycarbonate-polyether block copolymers (for example obtainable under the trade name Gambrane). Hollow fiber membranes with an asymmetric structure in which the lumen is surrounded by a fine-pored separation layer whereas the wall has pores of increasing size and/or open pores towards the outside are particularly suitable.

The external diameter of the hollow fiber membrane should not exceed 600 μm and should preferably be below 300 μm, particularly preferably below 200 μm. Hollow fiber membranes with a small diameter for example an internal diameter in the range of 50 to 100 μm, preferably 60 to 90 μm and a small wall thickness of for example 20 to 80 μm, preferably 40 μm are also suitable. The latter can be processed to form the catheters according to the invention with a particularly small overall diameter.

Adhesion to the reinforcement can be achieved by applying a thin layer of adhesive on the reinforcement and subsequently bonding it with the hollow fiber membrane. Suitable adhesives are reactive adhesives such as cyanoacrylate adhesives or polyurethane adhesives as well as adhesives containing solvent or adhesives that can be activated by radiation or heat-activated polymers or adhesives (hot-melt adhesives) such as ethylene/ethylacrylate copolymers, ethylene/vinylacrylate copolymers, polyamides, polyesters, polyisobutylene or polyvinylbutyrates and the membrane materials themselves. It is also possible to directly bond the reinforcement with the membrane by intimately contacting the hollow fiber membrane with the reinforcement and heating the reinforcement to a temperature above the melting temperature of the membrane material and/or above that of the reinforcement material when using a reinforcement made of plastic. This method is also suitable for activating a heat-activated adhesive. If a metallic reinforcement is used, it can be heated by feeding in electrical energy to the reinforcement. This method is particularly preferred since it was surprisingly found that during the melting process only the outer large-pore and/or open-pore contact zone between the membrane and reinforcement is sealed, but that the associated area of the separation layer is unchanged and thus remains open. Thus this type of bonding the hollow fiber membrane to the reinforcement only marginally reduces the exchange performance.

In order to secure the reinforcement of the catheter according to the invention it is also possible to apply a drop of a reactive polymer mixture to the front end i.e. the bent end of the catheter which further improves the adhesion between the membrane and reinforcement and seals any weak spots and leaks that may be present in the membrane in the bent portion. The adhesives mentioned above can also be used for this.

The liquid-impermeable zone in the area of the inlet and/or outlet of the catheter according to the invention can for example be made by immersing the hollow fiber membrane in a suitable polymer or polymer mixture or an adhesive which is then thermally or reactively hardened. However, it is preferable to heat these sections to a temperature near to the melting point of the membrane material so that the pores collapse in the hollow fiber membrane thus making the membrane practically impermeable to liquids. This is associated with a shrinkage of the hollow fiber membrane resulting in a reduction in the wall thickness and also a reduction in the internal diameter. An advantage of this procedure is that the reduction of the internal diameter of the hollow fiber membrane reduces the dead volume of the catheter according to the invention. This also reduces the dead time of a sample in the region of the inlet and/or outlet and allows a more rapid detection of a measured quantity such as a change in the glucose content in the tissue.

Heat treatment of the inlet and/or outlet section can be achieved by applying hot air, by partially inserting the catheter into a hot chamber or between two heated jaws or by the action of radiation. Infrared radiation is for example particularly suitable for precisely spatially shaping the catheter but care must be taken that radiation is only absorbed in the segments that are to be heated. This can be achieved by adding suitable substances that absorb radiation such as dyes or pigments.

The design of the inlet and/or outlet of the catheter according to the invention is selected such that both ends of the hollow fiber membrane are separated from one another. They can then be inserted and bonded in a known manner into recesses in a tube or in a carrier plate with miniaturized flow paths. The reinforcement can then be attached in a known manner in or on a tube or carrier plate with miniaturized flow paths for example in a recess or on a surface of the carrier plate, for example by gluing.

An example of an embodiment of the present invention is elucidated in more detail in the following.

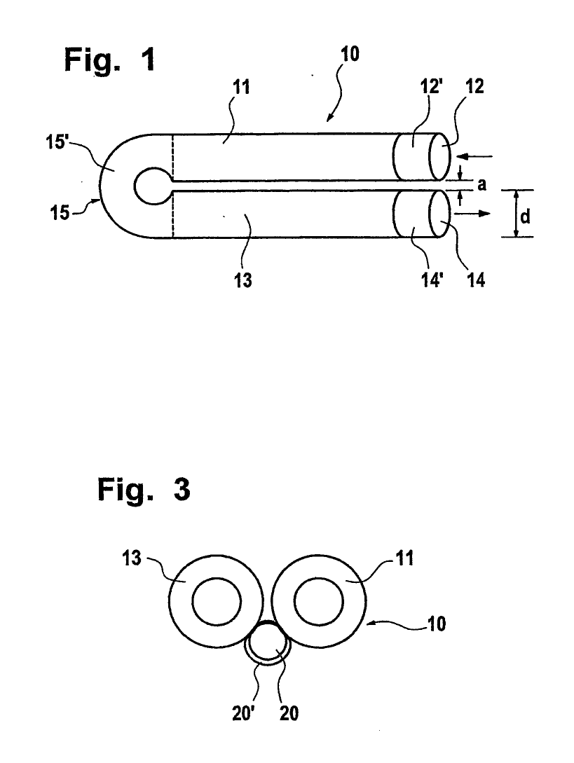

FIG. 1 shows a schematic, non-scale representation of a hollow fiber membrane for a catheter according to the invention after bending.

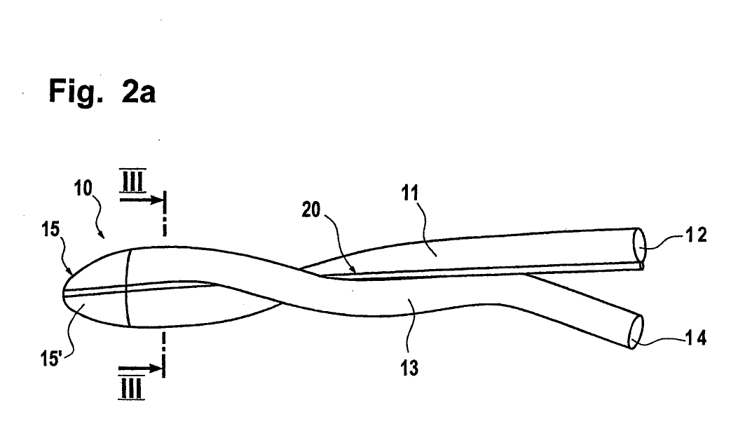

FIG. 2a shows the rear portion of a hollow fiber membrane from FIG. 1 with a reinforcement.

FIG. 2b shows the hollow fiber membrane from FIG. 1 with a reinforcement in its entirety.

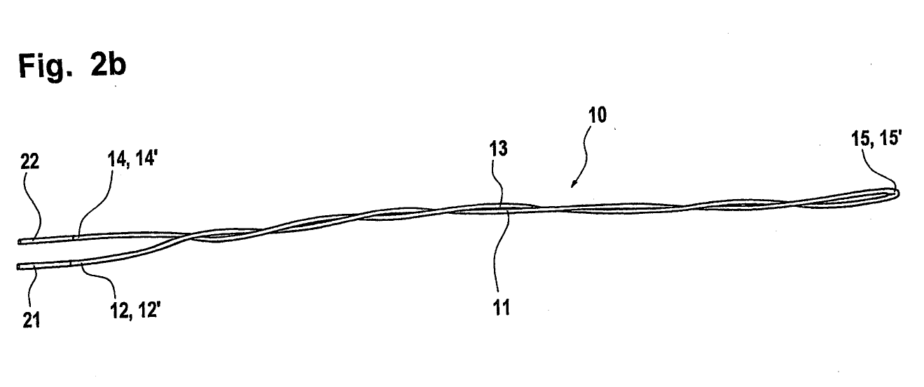

FIG. 3 shows a schematic cross-section through the hollow fiber membrane from FIGS. 2a and 2b.

1a. Bending the Hollow Fiber Membrane

A PAS hollow fiber membrane from Gambro Dialysatoren Co. Hechingen, Germany with an internal diameter of 214 μm and a wall thickness of 43 μm is cut to a length of 50 mm. A piece of hollow fiber membrane is placed centrally on an electrically heatable constantan wire having a diameter of 250 μm. The two ends of the hollow fiber membrane are loaded with a force of 1 mg. This wire is electrically heated. A current of 1.7 A flows over a period of 2×2 seconds from a current regulated power supply. The hollow fiber membrane is deformed and bent by the heated wire at the bearing site resulting in the formation of the two legs at an angle of 150 to 30°. The angle can be reduced to almost zero by carefully squeezing when the current is switched off in the second cycle.

FIG. 1 shows a diagram of such a U-shaped hollow fiber membrane 10 with two legs 11, 13 which each end in an inlet 12 or outlet 14 and a bent section 15. The direction of flow of the dialysis fluid is indicated by the arrows. The distance a between the legs 11 and 13 is less than 50% of the diameter d of the hollow fiber membrane 10. A liquid-impermeable area 12′, 14′ is located in the area of the inlet 12 and the outlet 14. Another liquid-impermeable area 15′ is located in the bent section 15 which is impregnated with an adhesive.

1b. Partially Automated Bending of a Hollow Fiber Membrane

A PAS hollow fiber membrane from Gambro Dialysatoren Co. Hechingen, Germany with an internal diameter of 214 μm and a wall thickness of 43 μm is cut to a length of 50 mm and placed on a support. The support has a narrow, straight recess in the middle in which an electrically heatable constantan wire having a diameter of 250 μm and oriented perpendicular to the surface is guided in a uniform movement at ca. 10 mm/s. A current of 1.7 A flows through the wire. During its forwards movement the wire strikes the middle of the hollow fiber membrane and carries it along. At the sides of the path of movement of the wire there are walls whose distance from the path of the wire decreases asymptotically to 500 μm. The transported hollow fiber bends in the desired manner under the influence of the wire heated by the current flow and the walls. The angle between the two legs can be reduced to almost zero by moving together two lateral jaws made of silicon at the moment when the current is switched off. The movement of the wire and the jaws and the current are automatically controlled. This readily allows a reproducible forming of the bend.

2. Manufacture of a Reinforcing Wire

A wire made of stainless steel (material No. 1.4301, Fe/Cr18/Ni10, degree of hardness: annealed, diameter: 0.05 mm) from Goodfellow Germany, Bad Nauheim, Germany, order No. FE225 110 was used for the reinforcement. The wire was coated with a thin layer of polyamide to improve adhesion. For this purpose the wire was pulled through a coating nozzle (diameter 300 μm) at a speed of 3.1 m/min and coated on all sides with a layer of a solution of 15% by weight Trogamid T3000, manufactured by Creanova, Marl, Germany, 7% by weight PVP Plasdone C-15, manufactured by ISP Technologies Inc. Wayne, USA and 78% by weight NMP (N-methyl-2-pyrrolidone) manufactured by Merck, Darmstadt, Germany. The wire coated in this manner is pulled through a water bath to wash out the solvent. A solid white layer is formed having a thickness of ca. 100 μm. After drying in air the coated reinforcing wire is ready to use.

3. Reinforcement of the Hollow Fiber Membrane

A hollow fiber membrane treated as described in section 1a. or 1b. is wound around an almost endless reinforcing wire treated as described in section 2 in such a manner that the two legs of the hollow fiber membrane are in close proximity and together form a winding with a pitch of 5 to 10 mm per winding. In this process the bend is pressed directly onto the wire while a length of ca. 3 mm at the beginning and end of the hollow fiber membrane stick out from the wire. An electrical current of 0.2 A is passed through the wire for a period of 3.5 seconds. The wire heated by the current melts the coating on the reinforcing wire and the outer portion of the hollow fiber membrane. After switching off the current, the wire cools and the hollow fiber membrane adheres tightly.

4. Finishing the Catheter

The hollow fiber membrane treated as described in 1a. or 1b. and 3. is cut into individual pieces. For this purpose the reinforcing wire is cut off directly in front of the bend and cut to a length of about 30 mm. The two open ends of the hollow fiber membrane are bonded into a tube having an internal diameter of 500 μm. Epoxide resin “UHU plus schnellfest”, UHU Vertrieb GmbH, Bühl/Baden, Germany is used as the adhesive. A length of about 4 mm at the two ends of the hollow fiber membrane are impregnated with adhesive and are thus made impermeable. UV hardening Dymax 1181-M, Dymax Europe GmbH, Frankfurt a. M., Germany is used as the adhesive. 2×20 mm remain as the permeable exchange length.

FIGS. 2a and 2b show diagrams of a hollow fiber membrane 10 that is reinforced as described in section 3, and wound around a reinforcing wire 20. The bent section 15 is connected to the reinforcing wire 20 whereas the inlet 12 and outlet 14 stick out from the reinforcing wire 20 over the length of the impermeable sections 12′ and 14′. Tubes 21 and 22 are also indicated into which the inlet 12 and the outlet 14 are glued in.

FIG. 3 shows a cross-section along the line III-III in FIG. 2a. It shows that the coating 20′ of the reinforcing wire 20 is melted and fused with the outer region of the hollow fiber membrane.

5. Measurement of the Mass Transfer Performance

Distilled water is allowed to flow through a catheter prepared as described in section 4 at a flow rate of 0.1 μl/min and the catheter is placed in a beaker containing 200 mg/dl glucose. A glucose content in the dialysis fluid of 199 mg/dl is measured at the outlet of the catheter.

6. Measurement of the Dead Time (95)

The term dead time (95) refers to the dead time up to reaching a signal change of 95%. A catheter prepared as described in section 4 is combined with a suitable flow refractive index measuring cell and distilled water is allowed to flow through the catheter at a flow rate of 0.1 μl/min. The catheter is alternately placed in beakers containing 200 mg/dl glucose and distilled water. The glucose concentration in the measuring cell follows the concentration in the beaker with a time delay. After subtracting the delay caused by the volume of the measuring cell and its feed tube, 252 seconds remain for the dead time (95) of the catheter.

1. Catheter with a hollow fiber membrane (10) bent essentially in a U-shape comprising two legs (11, 13) and a bent section (15), characterized in that the hollow fiber membrane (10) is heat-treated and/or treated with at least one solvent at least in its bent section (15) in order to obtain the smallest possible overall diameter of the catheter with an open lumen of the hollow fiber membrane.

2. Catheter as claimed in claim 1, characterized in that the distance (a) between the two legs (11, 13) is less than 50% of the diameter (d) of the hollow fiber membrane (10) and/or the overall diameter of the catheter is less than 2.5 times the diameter of the hollow fiber membrane.

3. Catheter as claimed in claim 1 or 2, characterized in that the U-shaped section of the hollow fiber membrane (10) is bent by 180°±5°.

4. Catheter as claimed in one of the previous claims, characterized in that the hollow fiber membrane (10) has a reinforcement (20).

5. Catheter as claimed in claim 4, characterized in that a wire, thread and/or braid are provided as the reinforcement (20) which is bonded to the two legs (11, 13) by for example gluing or welding.

6. Catheter as claimed in claim 4 or 5, characterized in that the reinforcement (20) consists of a metal, a metal alloy or a plastic.

7. Catheter as claimed in claim 5 or 6, characterized in that the wire consists of high-grade steel, a noble metal or monofil polymer fibers.

8. Catheter as claimed in claim 5 or 6, characterized in that the thread consists of polymer fibers.

9. Catheter as claimed in claim 5 or 6, characterized in that the braid consists of several thin metallic threads.

10. Catheter as claimed in one of the claims 5 to 9, characterized in that at least a particular site on the hollow fiber membrane (10) has at least one substance that absorbs radiation preferably dyes and/or pigments.

11. Catheter as claimed in one of the previous claims, characterized in that a liquid-impermeable zone (12′, 14′) is provided in the area of the inlet (12) and/or outlet (14) for the dialysis fluid.

12. Catheter as claimed in one of the previous claims, characterized in that the hollow fiber membrane has an asymmetric structure such that the lumen is surrounded by a fine-pored separation layer whereas the wall has pores which increase in size towards the outside.

13. Catheter as claimed in one of the previous claims, characterized in that the external diameter of the hollow fiber membrane (10) is less than 600 μm, preferably less than 300 μm and particularly preferably less than 200 μm.

14. Catheter as claimed in one of the previous claims, characterized in that the internal diameter of the hollow fiber membrane (10) is 50 to 100 μm, preferably 60 to 90 μm.

15. Catheter as claimed in one of the previous claims, characterized in that the wall thickness of the hollow fiber membrane (10) is 20 to 80 μm, preferably 40 μm.

16. Hollow fiber membrane for a catheter as claimed in one of the claims 1 to 15 which is essentially bent in a U-shape having two legs (11, 13) and a bent section (15), characterized in that the hollow fiber membrane (10) is heat-treated and/or treated with at least one solvent at least in its bent section (15) in order to obtain the smallest possible overall diameter of the catheter with an open lumen of the hollow fiber membrane.

17. Hollow fiber membrane as claimed in claim 16, characterized in that the distance (a) between the two legs (11, 13) is smaller than 50% of the diameter (d) of the hollow fiber membrane (10).

18. Method for producing a catheter with a hollow fiber membrane (10) which is essentially bent in a U-shape or for producing a hollow fiber membrane which is essentially bent in a U-shape, characterized in that the hollow fiber membrane (10) is heat-treated and/or treated with at least one solvent at least in the region to be bent.

19. Method as claimed in claim 18, characterized in that the hollow fiber membrane is treated by direct contact with an electrically heated wire and/or by the action of electromagnetic radiation.

20. Method as claimed in one of the claims 18 or 19, characterized in that commercial hollow dialysis fibers are used.

21. Method as claimed in one of the claims 18 or 19, characterized in that hollow dialysis fibers having a smaller diameter and/or smaller wall thickness than commercial hollow dialysis fibers are used.

22. Method as claimed in one of the claims 18 to 21, characterized in that in order to bond a reinforcement (20), firstly a thin layer of adhesive is applied to the reinforcement (20) which is then bonded to the hollow fiber membrane (10).

23. Method as claimed in claim 22, characterized in that reactive adhesives, solvent-containing adhesives, polymers or adhesives that can be activated by radiation or thermally activatable adhesives (hot-melt adhesives) are used for the bonding.

24. Method as claimed in one of the claims 18 to 21, characterized in that the reinforcement (20) is bonded directly to the hollow fiber membrane (10).

25. Method as claimed in claim 24, characterized in that the hollow fiber membrane (10) is intimately contacted with the reinforcement (20) and the reinforcement is heated to a temperature above the melting temperature of the membrane material and/or above that of the reinforcement material.

26. Method as claimed in one of the claims 18 to 25, characterized in that a drop of a reactive polymer mixture is applied to the front end of the hollow fiber membrane (10).

27. Method as claimed in one of the claims 18 to 26, characterized in that the hollow fiber membrane (10) in the area of the inlet (12) and/or outlet (14) of the catheter is impregnated with a hardenable polymer and the polymer is subsequently hardened.

28. Method as claimed in one of the claims 18 to 26, characterized in that the hollow fiber membrane (10) in the area of the inlet (12) and/or outlet (14) of the catheter is heated to near the melting point of the membrane material.

29. Method as claimed in claim 28, characterized in that the hollow fiber membrane (10) is heated by the action of radiation, preferably IR radiation.

30. Method as claimed in one of the claims 18 to 29, characterized in that the ends of the hollow fiber membrane (10) are inserted into a tube or a carrier plate with miniaturized flow paths and then glued in.

31. Method as claimed in claim 30, characterized in that the reinforcement (20) is attached or glued in or on a tube or a carrier plate with miniaturized flow paths.

32. Catheter with a hollow fiber membrane bent in a U shape which is produced by the method according to claim 18.
